(19) United States
(12) Patent Application Publication
(10) Pub. No.: US 2015/0369783 A1
(43) Pub. Date: Dec. 24, 2015

(54) ENVIRONMENT EVALUATION APPARATUS AND ENVIRONMENT EVALUATION METHOD FOR CALCULATING AMOUNT OF CARBON DIOXIDE EMISSION REDUCTION

(71) Applicants: KABUSHIKI KAISHA TOSHIBA, Tokyo (JP); TOSHIBA TEC KABUSHIKI KAISHA, Tokyo (JP)

(72) Inventors: Yusuke Hamada, Mishima-shi (JP); Akihiro Mizutani, Mishima-shi (JP)

(21) Appl. No.: 14/309,168

(22) Filed: Jun. 19, 2014

(51) Int. Cl. G01N 33/00

(52) U.S. Cl. CPC G01N 33/004; G01N 33/0062

(57) ABSTRACT

An environment evaluation apparatus comprises an interface and a controller. The interface receives the number of sheets when using the electronic data instead of printing them on the sheets. The controller calculates a carbon dioxide reduction amount as the reduction amount of carbon dioxide emission according to the number of sheets received by the interface, adds the calculated carbon dioxide reduction amount to the carbon dioxide reduction amount stored in a memory and then stores the sum in the memory.

### FIELD

Embodiments descried herein relate to a technology of measuring the amount of carbon dioxide emission reduction obtained by restraining use of sheets.

### BACKGROUND

A system is known which measures the contribution a user makes to reducing carbon dioxide emission by using a sheet after carrying out a page allocation (Ninl), by using both sides of a sheet or by reusing a color-erased sheet.

However, in the conventional system, the contribution a user truly makes to the reduction of carbon dioxide emission by using another display method without printing on a sheet.

### DESCRIPTION OF THE DRAWINGS

FIG. 1 is a diagram illustrating a configuration example of a server according to an embodiment;

FIG. 2 is a diagram illustrating an example of the data constitution of a table stored in an environment evaluation database;

FIG. 3 is a diagram illustrating a configuration example of a system according to embodiment 1;

FIG. 4 is a flowchart illustrating an example of the operations carried out by a server according to embodiments 1 and 2;

FIG. 5 is a diagram illustrating a configuration example of a system according to embodiment 2;

FIG. 6 is a diagram illustrating a configuration example of a system according to embodiment 3;

FIG. 7 is a diagram illustrating an example of the data constitution stored in a terminal management server according to embodiment 3; and

FIG. 8 is a flowchart illustrating an example of the operations carried out in embodiment 3.

### DETAILED DESCRIPTION

In accordance with one embodiment, an environment evaluation apparatus comprises an interface and a controller. The interface receives the number of sheets when using the electronic data instead of printing them on the sheets. The controller calculates a carbon dioxide reduction amount serving as the reduction amount of carbon dioxide emission according to the number of sheets received by the interface, adds the calculated carbon dioxide reduction amount to the carbon dioxide reduction amount stored in a memory and then stores the sum in the memory.

In an embodiment, a user using electronic data uses no sheets to output data and calculates the amount of the reduced carbon dioxide emission (hereinafter referred to as a carbon dioxide reduction amount). In this way, the contribution degree of the user consuming no sheets is measured. In an embodiment, in addition to the conventional Ninl setting, duplex printing or the use of a color-erased sheet, the carbon dioxide reduction amount is determined with taking the following content into consideration:

through the cooperation with a conference system using electronic data, the number of sheets which should have been used are saved by only using electronic data in a conference and are calculated as the carbon dioxide reduction amount;

a mobile scan mode in which the scanned data is sent to a mobile terminal device and the like is executed, thereby outputting no scanned data to the sheets or printing no data on the sheets, and therefore the saved number of sheets are calculated as the carbon dioxide reduction amount;

to make the calculated carbon dioxide reduction amount approximate to the actual carbon dioxide reduction amount, the power consumption of each terminal used by a user is converted into a carbon dioxide emission amount, and then the carbon dioxide reduction amount is offset by the carbon dioxide emission amount.

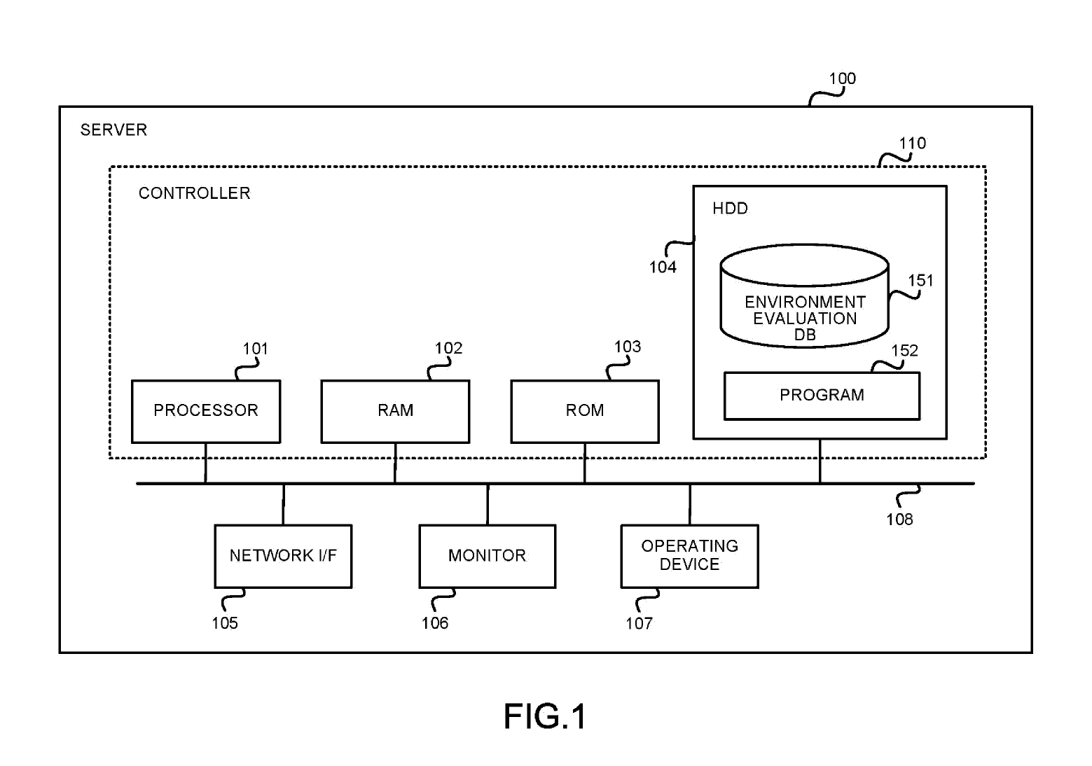

Embodiments of the present invention are described below in detail with reference to the accompanying drawings. FIG. 1 is a diagram illustrating the configuration of a server according to the embodiments. A server 100, which is a computer, comprises a processor 101, a RAM (Random Access Memory) 102, a ROM (Read Only Memory) 103, a HDD (Hard disk drive) 104, a network I/F (Interface) 105, a monitor 106 and an operating device 107, which are connected with each other via a bus line 108 to carry out the input and output of data and signals . Further, the structure including the processor 101, the RAM 102, the ROM 103, the HDD 104 and the bus line 108 for carrying out the input and output of data and signals among the aforementioned units constitute a controller 110.

The processor 101, which is, for example, an arithmetic device such as a CPU (Central Processing Unit), develops the program stored in the ROM 103 and the program 152 stored in the HDD 104 on the RAM 102 and executes the developed programs to cooperate with each piece of hardware to provide various functions. The RAM 102 is a main storage device, and the ROM 103 is a device for storing system programs in a non-volatile manner. The HDD 104 is an auxiliary storage drive for storing programs, data and the like in a non-volatile manner. The network I/F 105 is a LAN (Local Area Network) board for the communication control over the data received by the server 100 from an external device and the communication control over the data sent from the server 100 to the external device.

The monitor 106 and the operating device 107 (e.g. keyboard and mouse) constitute an operation and display section for the manager of the server 100 to perform a maintenance job. Further, the monitor 106 and the operating device 107 receive the display of the carbon dioxide reduction amount of each user and the current operation. An environment evaluation DB (database) 151 is constructed and a program 152 is stored in the HDD 104 in advance. Further, the environment evaluation DB 151 may be constructed in an external computer but not set in the server 100, and the server 100 makes a reference to the external computer.

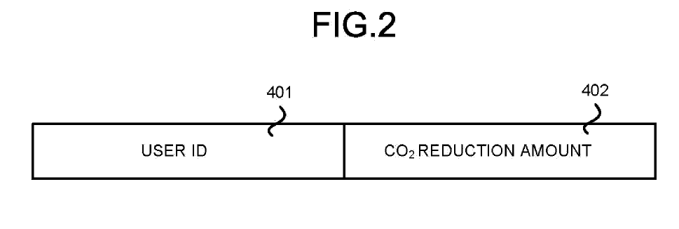

FIG. 2 is a diagram illustrating an example of the data constitution of a table constructed in the environment evaluation DB 151. The environment evaluation DB 151 includes a table in which each column of user ID 401 and the carbon dioxide reduction amount 402 are associated with each other. The user ID 401 is a column for storing the identification information of a registered user. The carbon dioxide reduction amount 402 is a column for totaling and storing the amount of the carbon dioxide emission reduced by the registered user. In the column of the carbon dioxide reduction amount 402, the value of amass unit system (e.g. gram) is stored.

Each embodiment in which the server 100 is used is described below.

### EMBODIMENT 1

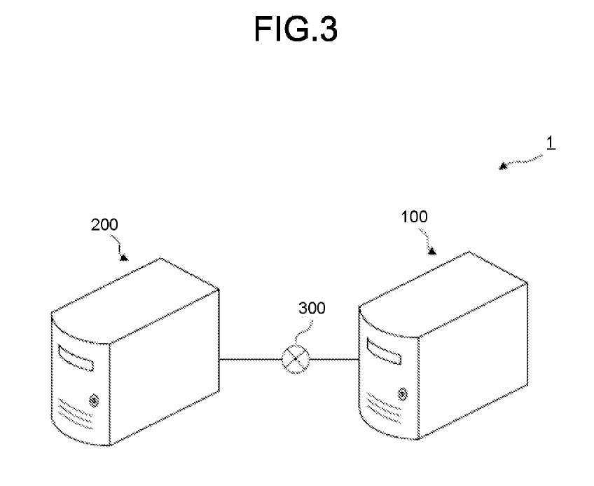

FIG. 3 is a diagram illustrating a configuration example of a system according to embodiment 1. A system 1 comprises the aforementioned server 100 and a conference management server 200 between which data transmission/reception is carried out via a network 300. The conference management server 200 is a server (computer) for supporting a paperless electronic conference. Identical to the server 100 in hardware constitution, the conference management server 200 comprises a processor, a RAM, a ROM, a HDD, a network I/F, a monitor, an operating device and a bus line for connecting the aforementioned units. If the conference management server 200 receives the registration of a conference scheduled to be held, then the conference management server 200 assigns the conference with an automatically numbered conference ID, associates the following information with the conference ID and then stores the associated information in a memory. Further, the conference management server 200 also stores the entity file of data of the material to be used.
holding date and holding place
the identification information of the conference sponsor the identification information of a presenter, the identification information of all the participants participating in the presentation
the file name (full pathname) of data of the material to be used

In a case where there are a plurality of subjects and a plurality of presenters in a conference, the number of a presenter of copies of presenter identification information and the number of a presenter of copies of participant identification information are stored.

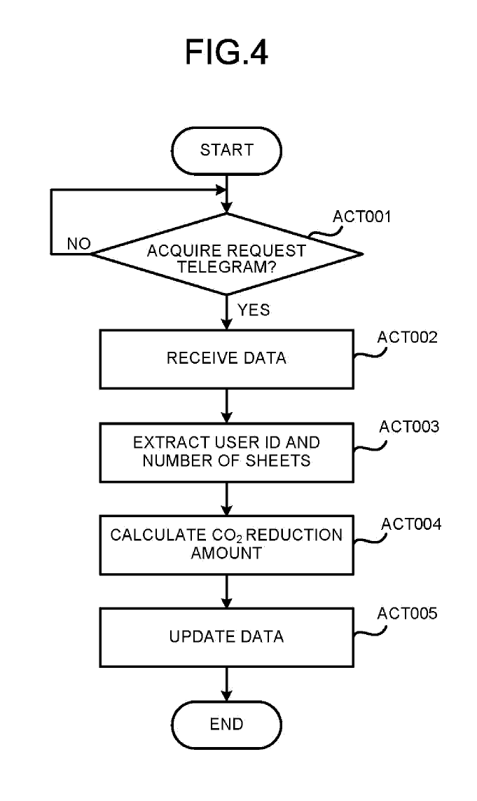

The conference sponsor using the conference management server 200 displays electronic data with a projector or a big monitor to progress the conference, using no printed sheets. The server 100 calculates the total carbon dioxide reduction amount obtained from all the presenters after the conference is ended or at a specific moment and adds the calculated amount to the totaled value of each user registered in the environment evaluation DB 151. The operations carried out by the server 100 at this time are shown in the flowchart of FIG. 4. The operations shown in FIG. 4 are realized by developing and executing the program 152 by the processor 101 on the RAM 102 and cooperating with each piece of hardware.

The processor 101 enters a standby state until a processing request telegram is received from the conference management server 200 (loop of NO in ACT 001). If the request telegram is received (YES in ACT 001), the network I/F 105 receives data containing a user ID and number of sheet (ACT 002). If there are a plurality of presenters, the network I/F 105 receives a presenter number of copies of the data. The processor 101 extracts the user ID and the number of sheet from the received data and temporarily stores the extracted user ID and number of sheet in the RAM 102 (ACT 003).

Herein, the user ID is the identification information of a user giving a presentation and becomes a user ID registered in the environment evaluation DB 151. The number of sheet is the number of sheets when using electronic data instead of printing then on the sheets. In other words, the number of sheets is the number of sheets on the assumption that the sheets are used in a conference and the number of the pages of data which should have been output on the sheets.

As no printed sheets are actually used, it is needed to convert conference material files into the number of sheet. In this example, the conference management server 200 calculates a number of sheets by converting each page that can be previewed using an application capable of displaying conference material files into one sheet . Further, the conference management server 200 calculates a total number of sheets by taking copies into consideration and sends the total number of sheets to the server 100. For example, if the number of sheets (number of pages) of electronic data material files is 5, and the number of the participants, including presenters, is 4, then the total number of sheets is 20 (5*4).

The number of sheets is calculated in the example by the conference management server 200; however, the number of sheets may also be calculated by the server 100. In this case, the network I/F 105 receives the identification information of a presenter, the identification information of all the participants participating in the presentation presenters (or the number of the participants) and the number of pages of the material files used in the presentation (or entity files of the material files) , and the processor 101 calculates the number of sheets using the received data.

The processor 101 calculates a carbon dioxide reduction amount based on the number of sheets (ACT 004). As a method for calculating the carbon dioxide reduction amount based on a paper medium, it may adopt a pre-determined existing mathematical formula, or the carbon dioxide reduction amount may be exported from a table in which number of sheets and carbon dioxide reduction amount are associated with each other in advance and which is stored in the HDD 104.

The processor 101 updates the carbon dioxide reduction amount stored in the environment evaluation DB 151 corresponding to the user ID (ACT 005). ACT 005 is described next. The processor 101 retrieves the environment evaluation DB 151 using the user ID extracted in ACT 003 and acquires the current totaled value of the carbon dioxide reduction amount of the user ID. The processor 101 adds the carbon dioxide reduction amount calculated in ACT 004 to the retrieved carbon dioxide reduction amount (totaled value) and updates the carbon dioxide reduction amount stored in the environment evaluation DB 151 corresponding to the user ID with the obtained addition value.

If the number of sheets used by each user can be obtained from an image forming apparatus and the like, a carbon dioxide emission amount can be calculated based on the number of sheets used, and then the carbon dioxide reduction amount of the user stored in the environment evaluation DB 151 is updated with the calculated carbon dioxide emission amount. In this case, the following processing is carried out: subtracting the calculated carbon dioxide emission amount from the carbon dioxide reduction amount stored in the environment evaluation DB 151. Further, the carbon dioxide reduction amount may also be calculated by taking the size of the sheets into consideration in ACT 004. In this case, the network I/F 105 also receives sheet size information in ACT 002 and, the processor 101 calculates the carbon dioxide reduction amount in ACT 004 by integrating a coefficient corresponding to the sheet size with the received sheet size information.

Further, in the aforementioned embodiment, only the totaled value of a carbon dioxide reduction amount is stored for each user in the environment evaluation DB 151. As another embodiment, the user ID may be associated with the carbon dioxide reduction amount calculated in ACT 004 into a set, and then the set is cumulatively stored in the environment evaluation DB 151. In this case, instead of updating the carbon dioxide reduction amount stored in the environment evaluation DB 151, a new record is registered in ACT 005. When the totaled value is needed, the processor 101 retrieves the environment evaluation DB 151 using the user ID and then calculates the sum of the retrieved recorded carbon dioxide reduction amount.

Thus, the amount of the carbon dioxide reduced by replacing the printing of data on sheets with the display of electronic data in a conference or consultation is taken into consideration, thus, a more accurate carbon dioxide reduction amount can be obtained.

### EMBODIMENT 2

The cooperation with an image forming apparatus is described in embodiment 2. In recent years, the image forming apparatus includes one which wirelessly sends scanned image data to a portable terminal device such as a smart phone instead of outputting the scanned image data to a sheet. The portable terminal device receiving the image data visually recognizes the image data without printing the image data on a sheet. In embodiment 2, through the cooperation with such an image forming apparatus, the carbon dioxide reduction reduced amount is calculated by taking the number of sheets which should have been printed into consideration.

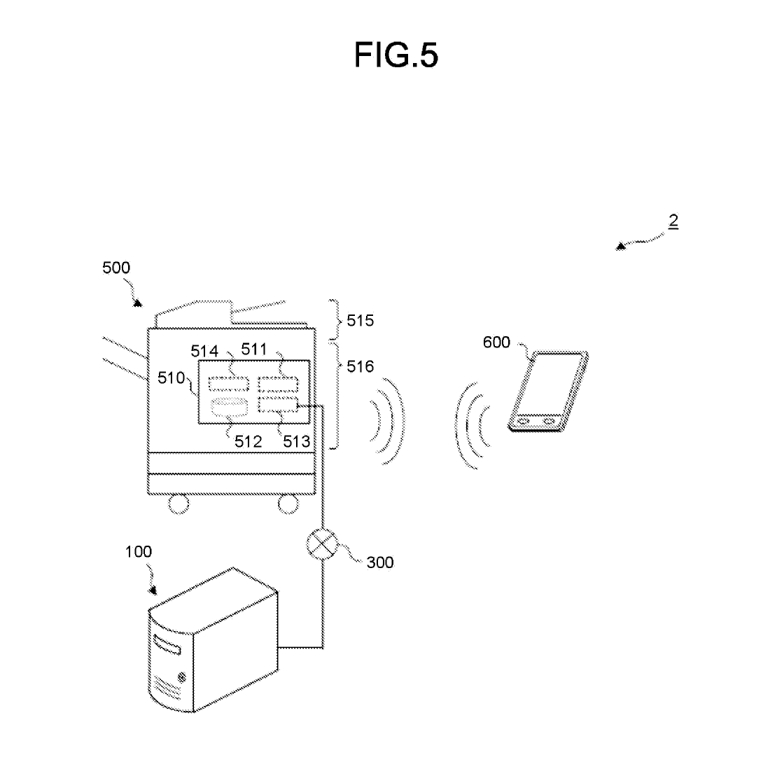

The configuration example of the system of embodiment 2 is shown in FIG. 5. A system 2 comprises the aforementioned server 100 and an image forming apparatus 500, which are connected with each other via the network 300. Further, although not shown in FIG. 5, the conference management server 200 describe in embodiment 1 may be connected with the network 300.

The image forming apparatus 500 comprises a control board 510, a scanning section 515 and a printing section 516. The scanning section 515, which includes a light-emitting section, an optical system and a CCD image sensor, scans and reads an original sheet placed by a user at a specific position. That is, the light-emitting section irradiates the original sheet, and the CCD image sensor receives the light with an optical system and converts the image into electronic data. The printing section 516 includes a transfer device for transferring an image onto a sheet, a fixer for heating the sheet on which the image is transferred to fix the image on the sheet and a conveyance section for conveying the sheet to each unit inside the apparatus.

The control board 510, which is a board for totally controlling each piece of hardware inside the image forming apparatus 500, comprises a processor 511, a HDD 512, a network I/F 513 and a RAM 514. The processor 511, the HDD 512 and the RAM 154 are equivalent to the processor 101, the RAM 102 and the HDD 104 in the server 100. In addition to a LAN board for carrying out wired communication with the server 100, the network I/F 513 also includes a device in accordance with short range radio communication standard. Thus, the network I/F 513 is capable of carrying out data transmission/reception with a portable terminal device 600 in a wireless manner. The portable terminal device 600 is the portable computer (e.g. smart phone, notebook computer or tablet computer) held by a user who is operating the image forming apparatus 500 currently.

When using the image forming apparatus 500, a log in authentication is carried out, and the user can use the image forming apparatus 500 only in a case where the authentication is conform. The image forming apparatus 500 switches to a mobile scan mode according to a specific operation of the user such that the scanned data is wirelessly sent to the portable terminal device 600 instead of being printed and output on a sheet. The image forming apparatus 500 acquires the identification information (user ID) of the user who is currently using the image forming apparatus and passed through the authentication processing and acquires the number of sheets scanned by the user in the mobile scan mode. The processor 511 of the image forming apparatus 500 associates the acquired user

ID with the acquired number of sheets and then stores the associated data in the HDD 512 in the job unit. Further, the acquired user ID and the acquired number of sheets may also be associated with time information, such as the current date and time.

The processor 101 of the server 100 executes the operations shown in the flowchart of FIG. 4 exemplified in embodiment 1. That is, if the processor 101 of the server 100 acquires a request telegram from the image forming apparatus 500 (YES in ACT 001), then the data in which user ID and number of sheets are associated is acquired (ACT 002), and then, the user ID and the number of sheets are extracted from the data (ACT 003). Further, the processor 101 calculates a carbon dioxide reduction amount based on the number of sheets (ACT 004), adds the calculated carbon dioxide reduction amount to the carbon dioxide reduction amount (totaled value) in the environment evaluation DB 151 and then updates the carbon dioxide reduction amount corresponding to the user ID (ACT 005).

Further, the user ID may be associated with the carbon dioxide reduction amount calculated in ACT 004 into a set, as described in embodiment 1, and then the set is directly and cumulatively stored in the environment evaluation DB 151. In this case, instead of updating the carbon dioxide reduction amount corresponding to the user ID, a new record is registered in ACT 005. The processing carried out when the totaled value is needed is the same as that carried out in embodiment 1.

In this way, the amount of the carbon dioxide reduced without outputting a corresponding number of sheets which should have been output from the image forming apparatus 500 is taken into consideration, thus, a more accurate carbon dioxide reduction amount is obtained.

### EMBODIMENT 3

In embodiment 3, the power consumption of each terminal (client computer) used by a user is converted into a carbon dioxide emission amount, and the obtained carbon dioxide emission amount is subtracted from the totaled value of the carbon dioxide reduction amount stored in the environment evaluation DB 151. In this way, the carbon dioxide reduction amount is more approximate to an accurate value.

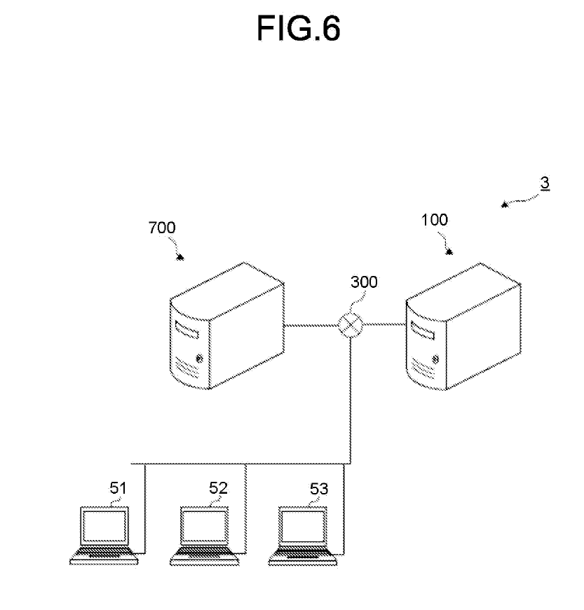

FIG. 6 shows a configuration example of a system according to embodiment 3. A system 3 comprises client terminals 51-53, a terminal management server 700 and the server 100 among which data transmission/reception is carried out with one another via the network 300. Further, although not shown in FIG. 6, the conference management server 200 described in embodiment 1 or the image forming apparatus 500 described in embodiment 2 may also be connected with the network 300.

The client terminals 51-53 are computers daily used by users. The terminal management server 700 is a server (computer) for managing the usage condition and the like of the client terminals 51-53. Identical to the server 100 in hardware constitution, the terminal management server 700 comprises a processor, a RAM, a ROM, a HDD, a network I/F, a monitor, an operating device and a bus line. The terminal management server 700 respectively stores the power consumption of the client terminals 51-53.

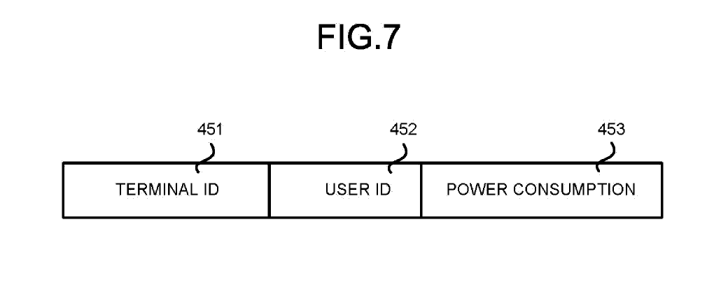

FIG. 7 is a configuration example of a table in a DB managed by the terminal management server 700. The terminal management server 700 stores a table in which each column of terminal ID 451, user ID 452 and power consumption 453 is associated with each other. The terminal ID 451 is a column for storing the identification information of the client terminals 51-53. The user ID 452 is a column for storing the identification information of the user of each client terminal, in which user IDs are stored like the user ID 401 shown in FIG. 2. The power consumption 453 presents the totaled value of power consumption of each client terminal. The terminal ID 451 and the user ID 452 are changed when performing system maintenance and are not updated when the system is running. On the other hand, the power consumption 453 is updated data accumulated within a unit time (e.g. per 15 min), and the updated power consumption data is reset to be 0 after being sent to the server 100. The terminal management server 700 acquires the power consumption of each of the client terminals 51-53 within each unit time, adds the acquired power consumption to the value stored in the power consumption 453, and updates the power consumption 453 with the sum.

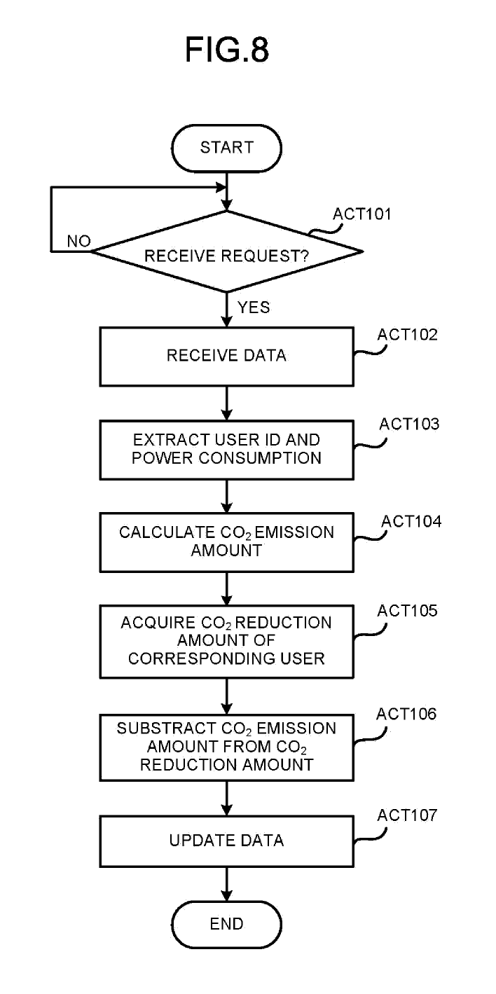

FIG. 8 is a flowchart illustrating an example of the operations carried out by the server 100 in embodiment 3. Like in embodiments 1 and 2, the operations shown in FIG. 8 are realized in the following way: the processor 101 develops the program 152 on the RAM 102 and then executes the program 152 to cooperate with each piece of hardware.

The processor 101 enters a standby state until the network I/F 105 receives a processing request telegram from the terminal management server 700 (loop of NO in ACT 101). If the request telegram is received (YES in ACT 101), the network I/F 105 receives the data in which user ID and power consumption are associated from the terminal management server 700 (ACT 102). When a plurality of user IDs are registered in the terminal management server 700, the network I/F 105 receives the aforementioned data for the number of records. The processor 101 extracts the user ID and the power consumption from the data received and temporarily stores the extracted user ID and power consumption in the RAM 102 (ACT 103).

The processor 101 calculates a carbon dioxide emission amount according to the power consumption (ACT 104). As the method for calculating the carbon dioxide emission amount according to the power consumption, it may be a predetermined existing mathematical formula, or the carbon dioxide emission amount is exported from a pre-defined table (a table in which a power consumption is associated with a carbon dioxide emission amount).

The processor 101 retrieves the environment evaluation DB 151 using the user ID extracted in ACT 103 and acquires the current carbon dioxide reduction amount (totaled value) of the user ID (ACT 105). The processor 101 subtracts the carbon dioxide emission amount calculated in ACT 104 from the retrieved carbon dioxide reduction amount (ACT 106). The processor 101 put units in order, if needed, and then performs the subtraction operation. The processor 101 updates the carbon dioxide reduction amount stored in the environment evaluation DB 151 corresponding to the user ID with the value obtained from the subtraction (ACT 107).

Further, as described in embodiments 1 and 2, in a case where a user ID and a current carbon dioxide reduction amount, instead of a totaled value, are stored cumulatively, the processor 101 associates the user ID with the carbon dioxide emission amount calculated in ACT 104 into a set and then cumulatively stores the set in the environment evaluation DB 151. In this case, no subtraction processing is carried out in ACT 106, and instead of updating data, a new record is registered in ACT 107. If the totaled value data in which the carbon dioxide emission amount is taken into consideration is needed, the processor 101 retrieves the environment evaluation DB 151 using the user ID, adds up the carbon dioxide reduction amount of the records hit by the retrieval and then subtracts the hit carbon dioxide emission amount from the sum of the recorded carbon dioxide reduction amounts.

The aforementioned embodiments 1-3 can be combined each other. Further, the server 100 may be provided as an environment evaluation apparatus. The functions of each server are not limited to be divided in the way described in the aforementioned embodiments. Further, the functions dispersedly provided in each server may be integrated in one or few servers.

According to the aforementioned embodiments, the degree of contribution a user using no sheets makes to environment can be accurately calculated by calculating a carbon dioxide reduction amount according to the number of the used sheets.

In the embodiments, any of the processes described above can be accomplished by a computer-executable program, and this program can be embodied in a non-transitory computer-readable memory device. In the embodiments, the memory device, such as a magnetic disk, a flexible disk, a hard disk, an optical disk (CD-ROM, CD-R, DVD, and so on), an optical magnetic disk (MD and so on) can be used to store instructions for causing a processor or a computer to perform the processes described above. Furthermore, based on an instruction in the installed program, an OS (operating system) of the computer, or MW (middleware software), such as database management software or network, may execute one or more parts of the processes described above to realize the embodiments.

While certain embodiments have been described, these embodiments have been presented by way of example only, and are not intended to limit the scope of invention. Indeed, the novel apparatus and methods described herein may be embodied in a variety of other forms; furthermore, various omissions, substitutions and changes in the form of the apparatus and methods described herein may be made without departing from the spirit of the inventions. The accompanying claims and their equivalents are intended to cover such forms or modifications as would fall within the scope and spirit of the inventions.

What is claimed is:

1. An environment evaluation apparatus, comprising:
an interface configured to receive the number of sheets when using the electronic data instead of printing them on the sheets; and
a controller configured to calculate a carbon dioxide reduction amount as the reduction amount of carbon dioxide emission according to the number of sheets received by the interface, add the calculated carbon dioxide reduction amount to the carbon dioxide reduction amount stored in a memory and then store the sum in the memory.

2. The environment evaluation apparatus according to claim 1, wherein
the memory associates the identification information of a user with a carbon dioxide reduction amount and then stores them;
the interface receives the data in which user identification information and the number of sheets are associated; and
the controller adds the calculated carbon dioxide reduction amount to the carbon dioxide reduction amount obtained by retrieving the memory using the identification information of the user, associates the resulting carbon dioxide reduction amount with the identification information of the user, and then stores the associated data in the memory.

3. The environment evaluation apparatus according to claim 2, wherein
the interface further receives data in which identification information of the user and the power consumption of the computer used by the user are associated; and
the controller calculates a carbon dioxide emission amount according to the power consumption, subtracts the calculated carbon dioxide emission amount from the carbon dioxide reduction amount obtained by retrieving the memory using the identification information of the user, associates the resulting carbon dioxide reduction amount with the identification information of the user, and stores the associated data in the memory.

4. The environment evaluation apparatus according to claim 1, wherein
the interface receives the number of sheets from a computer for managing a conference in which electronic material data is used.

5. The environment evaluation apparatus according to claim 1, wherein
the interface receives the number of sheets from an image forming apparatus which sends image data obtained by scanning an original sheet to an external portable device.

6. An environment evaluation apparatus, comprising:
an interface configured to receive the number of sheets when using the electronic data instead of printing them on the sheets; and
a controller configured to calculate a carbon dioxide reduction amount as the reduction amount of carbon dioxide emission according to the number of sheets received by the interface and store the calculated carbon dioxide reduction amount in a memory.

7. An environment evaluation method, including:
receiving the number of sheets when using the electronic data instead of printing them on the sheets;
calculating a carbon dioxide reduction amount as the reduction amount of carbon dioxide emission according to the received number of sheets; and
adding the calculated carbon dioxide reduction amount to the carbon dioxide reduction amount stored in a memory and then storing the resulting sum in the memory.

8. The environment evaluation method according to claim 7, wherein
the memory associates the identification information of a user with a carbon dioxide reduction amount and then stores the associated data;
further including:
receiving the data in which the user identification information and the number of sheets are associated,
adding the calculated carbon dioxide reduction amount to the carbon dioxide reduction amount obtained by retrieving the memory using the identification information of the user, associating the resulting carbon dioxide reduction amount with the identification information of the user, and storing the associated data in the memory.

9. The environment evaluation method according to claim 7, including:
receiving the number of sheets from a computer for managing a conference in which electronic material data is used.

10. The environment evaluation method according to claim 7, including:
receiving the number of sheets form an image forming apparatus which sends image data obtained by scanning an original sheet to an external portable device.
